(19) United States
(12) Patent Application Publication
(10) Pub. No.: US 2003/0032035 A1
(43) Pub. Date: Feb. 13, 2003

(54) Microfluidic device for analyzing nucleic acids and/or proteins, methods of preparation and uses thereof

(75) Inventors: Francois Chatelain, Le Chevalon De Voreppe (FR); Felix W. Frueh, Darnestown, MD (US)

Correspondence Address: Lerner, David, Littenberg, Krumholz & Mentlik, LLP, 600 South Avenue West, Westfield, NJ, 07090, US

(73) Assignee: COMMISSARIAT A L'ENERGIE ATOMIQUE, Paris (FR)

(21) Appl. No.: 10/139,100

(22) Filed: May 3, 2002

Related U.S. Application Data
(60) Provisional application No. 60/288,526, filed on May 3, 2001.

(51) Int. Cl. C12Q 1/68; G01N 33/53; G01N 33/542; C12M 1/34

(52) U.S. Cl. 435/6; 435/7.9; 435/287.2

(57) ABSTRACT

The invention relates to a microfluidic device for nucleic acid and/or protein analysis, wherein said device comprises a capillary on the inner surface of which is attached an array of at least two reagents. Also encompassed by the invention is such a capillary. In addition, the invention concerns methods for attaching at least two reagents on the inner surface of a capillary, as well as methods using a microfluidic device and enabling a multiplex analysis of nucleic acids and/or proteins to be performed.

The present invention claims priority from U.S. provisional application No. 60/288,526 filed on May 3, 2001, the text of which is hereby incorporated by reference.

### FIELD OF THE INVENTION

The present invention relates to a microfluidic device, methods for preparing said device, and uses thereof for nucleic acid and/or protein analysis. More specifically, the present invention provides a microfluidic device, as well as a fast and effective method using said device, to detect nucleotide polymorphisms and mutations. Said device and method can also be used to measure gene expression levels and to analyze proteins, i.e., their structure, function, concentration, lability, and other properties regarding proteins.

The microfluidic device according to the invention allows the implementation of a high-throughput method for genotyping, gene expression analysis, and proteomics, said method also forming part of the present invention.

### BACKGROUND AND PRIOR ART

The intense efforts to sequence and map the human genome have provided us with a draft sequence of the human genome and soon the entire genome will be known. One of the most valuable assets associated with the sequencing effort was the discovery of genetic diversity, based on single- or poly-nucleotide polymorphisms. The current draft of the genome is providing a reference sequence to which such sequence variations, polymorphisms, and mutations, can be aligned and compared. As an example, in the medical field, such comparative analyses between a reference nucleotide sequence and a sequence obtained from a person displaying a disorder or a disease, or having a predisposition to such a disorder or a disease, are useful for the study, diagnosis and treatment of said human disorder or disease.

Gene expression analysis has become a widely used technique to investigate the biological effect of environmental variations resulting from, for instance, the presence of drugs, chemicals, nutrients, or physical changes such as temperature variations, light exposure changes, or drought on plants, or other changes having a biological and/or physiological effect.

Encompassed in the technical field of the present invention are microarrays. In this area, methods exist for synthesizing or attaching hundreds or thousands of reacting species to a solid and generally planar surface (1, 2). These species can either participate in simple binding reactions or in more complicated multi-step reactions (3, 4). In practice, limitations arise from the planar format, said limitations including for instance large sample size requirement, sample introduction difficulties, sub-optimal sensitivity, and often sub-optimal rates of hybridization resulting in misinterpretation of the results.

Microfluidic devices allow materials to be transported through capillary networks by the application of pressure or electro-kinetic forces to the device channels. Such devices can be useful for parallel analysis (i.e., multiplex analysis), as well as high throughput chemical reactions and screening. Nevertheless, in practice, performing multiplex reactions requires universal means to introduce the sample, said means being often inefficient, cumbersome and complicated because of cross contamination. Performing multiplex reactions with the same chemical reagents is possible since those reagents are housed in reagent wells on the microfluidic device so that numerous sample species can be exposed to said reagents, in parallel or in series. However, both the microfluidic devices and the samples can be fouled by carry over due to wall adsorption of reactants and analytes. Moreover, such devices are not suited for multiplex reactions using numerous reactants when numerous and various reactant species present in the reagent mix need to be equally and simultaneously available to the samples.

Therefore, there is a need in the art for devices and methods enabling nucleotide sequence variations, polymorphisms and mutations, as well as protein amounts, enzyme activities, binding reaction kinetics, to be monitored, said devices and methods being easy to use and to implement, respectively, and being also rapid and efficient.

The present invention satisfies this need by providing tools that efficiently combine the merits of both microarrays and microfluidic devices, while minimizing the shortcomings of each.

### SUMMARY OF THE INVENTION

The present invention relates to a microfluidic device for nucleic acid and/or protein analysis, wherein said device comprises a capillary on the inner surface of which is attached an array of at least two reagents.

The invention also provides a method for preparing such a microfluidic device, by attaching reagents on the inner surface of a capillary, said method comprising either the step of sequentially immobilizing pre-synthesized reagents on said surface, or the step of synthesizing in situ said reagents.

The present invention also concerns a method for analyzing nucleic acids and/or proteins. This method enables a multiplex analysis to be performed.

Thus, the device and methods according to the present invention provide tools to detect nucleotide sequence variations, and to analyze differential gene expression under varying environmental conditions.

The present invention is also useful in proteomics. This includes the entire area of protein analysis: for instance, the microfluidic device according to the invention can be used to investigate protein amounts in a biological sample, protein-protein interactions, protein-nucleic acid interactions, and protein functions such as metabolic activities.

### BRIEF DESCRIPTION OF THE DRAWINGS

FIG. 1 schematically illustrates the body structure and dimensions of a microfluidic device according to the invention, said device incorporating the serial attachment of binding species or reacting moieties immobilized or synthesized in situ.

FIG. 2 schematically illustrates reagent attachment and detection geometries for a flow-through capillary useful for synthesis and detection. In this figure, DNA synthesis and the required chemicals are depicted.

FIG. 3 is a schematic representation of the serial steps of photo-deprotection during a cycle of immobilization of oligonucleotides on the surface of a capillary. In this Figure, R is diisopropyl moiety ; R1, R2, and R3 are permanently protected bases (A, G, C, or T); and BG is the photolabile protective group. The “Mask” is a cover that permits to hide parts of the capillary, and to expose to light only the desired area.

FIG. 4 depicts three methods of sample introduction into a device according to the invention, after the step of in situ synthesis. FIG. 4A shows the use of pressure (i.e., applied by pumps or syringes) to move the liquid into and within the capillary. FIG. 4B shows the use of electro-kinetic forces: solutions are moved under an electrical field spanning from the reservoir to the sample containers. FIG. 4C shows the use of capillary forces to move solutions.

FIG. 5 illustrates the use of an excitation source along the length of a device according to the invention, said excitation source being associated to a detector [here a photomultiplicator (PMT)] that monitors light emission, either at a single point or along the length of the device. In this illustration, the device is moved whereas the light source is fixed.

FIG. 6 illustrates an embodiment of the processed signal when the present invention is applied to gene expression. The signals generated by hybridizing target molecules (i.e., molecules contained in the sample that is introduced into the capillary) to the probes (i.e., reagent molecules synthesized in situ or immobilized in the capillary) are measured and the output graphically represents the relative intensity from one probe to another. Based on the intensity of gene expression analysis, a “genetic barcode” can be generated, said barcode being specific to the sample.

FIG. 7 corresponds to images illustrating the results obtained by hybridizing oligonucleotide probes CP-NH2 and 261A-NH2 with fluorescent oligonucleotide target CPc-Cy3 (see Example 1). FIG. 7 A corresponds to the following image analysis conditions: data-acquiring time of 50 msec; magnification 20×; panel 1: full match; panel 2: negative control. FIG. 7B illustrates hybridization of probe CP-NH2 (capillary α, above) or probe 261A-NH2 (capillary β, below) with the CPc Cy3 target under the following conditions: magnification 10×; panel 1: data-acquiring time of 50 msec; panel 2: data-acquiring time of 20 msec.

### DETAILED DESCRIPTION OF THE PREFERRED EMBODIMENTS

According to the invention, by “feature”, “reagent”, “reactant”, or “compound” is meant a biochemical or a chemical including, without any limitation, an oligonucleotide, a peptide, a polysaccharide, as well as other molecules that can react with the species to analyze (sample). In the present context, at least two reagents are simultaneously used, corresponding to an “array” of features, each feature being assigned to a specific area within the flow-through capillary of the invention.

By “array of features” is meant a collection of features, said collection comprising at least two features, and being spatially organized. In the context of the invention, said array is located on the inner surface of a capillary.

By “at least two reagents” is meant herein that the upper limit Nₘₐₓ of the number of reagents that can be attached to the inner surface of a given capillary can be calculated using the following formula:

Nₘₐₓ=L_{c}/Wₛₐ

wherein L_{c} is the capillary length and Wₛₐ is the width of the specific area to which one reagent is assigned, said width being the same for all the reagents.

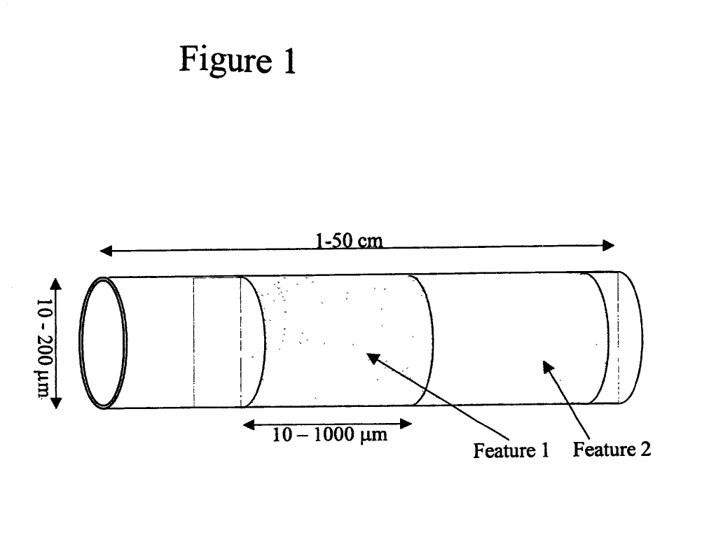

By “spatially organized” is meant that each feature comprised in said array is assigned to a specific area of said capillary. For instance, said area is a section of the capillary as illustrated in FIG. 1.

According to the invention, by “sample” or “sample species” or “analyte” is meant the biochemical or chemical molecule, or mix of molecules, to analyze, said molecule or mix of molecules including, without any limitation, nucleic acids, proteins, and the like. Said molecule or mix of molecules also corresponds to a “target” or a mix of “targets”. To facilitate the reading of the present specification, “target” will be equally used to refer to a specific target or to a mix of targets.

Terms “peptide”, “oligopeptide”, “polypeptide”, and “protein” are equally used herein to refer to a molecule consisting of a sequence of amino acids, whatever its length is, said sequence being equally encoded by a “nucleic acid”, a “nucleotide” molecule, an “oligonucleotide” or a “polynucleotide” molecule, said “nucleic acid” being constituted by a sequence of nucleotides, whatever its length is. Sequences of amino acids encompassed within the present invention are substituted (such as glycopeptides), or not.

By “capillary” is meant a device, and more specifically, a tube or a channel having a diameter less than 1 mm. Examples of diameters of a capillary according to the invention are given in Table 1 (see below).

By “device” or “system” is meant herein a mechanism the function of which generally refers to at least one operation necessary for the good working of a whole including said device, said whole being for instance a machine or an apparatus. For instance, said device is a capillary as defined above. In another embodiment, said device consists of said whole, and is for instance said machine or said apparatus. A skilled artisan, relying on the context in which terms “device” and “system” are used in the specification and the claims, will easily and undoubtedly understand which meaning should be given to said terms.

By “functionalize” is meant that an active site either exists naturally on an entity (it is then a potentially active site that has to be activated) or is generated on said entity by methods well-known in the art, some of them being described herein (for instance, a method of activation of a capillary surface using a synthetic linker that provides a reactive site for covalent attachment of reagents on said surface, see below the part of the Detailed Description dealing with the method of attachment according to the invention). By “functionalizing” said entity is meant rendering it functional, i.e., reactive.

The term “entity” has a general meaning, encompassing for instance molecules, devices as defined above, parts of said molecules or devices, and others. In one embodiment, said entity refers to the inner surface of a capillary according to the present invention.

By “deprotection” is meant the removal of a chemical protective group from a reactive moiety. The skilled artisan will appreciate the kind of “deprotection” it is herein dealt with in the view of the context, and relying on the Examples set forth below.

By “chemical building block” is meant a structural unit of a molecule, the sequence or chain formed by all said structural units leading to said molecule. More specifically, when said molecule is a chemical, said “chemical building block” is an atom, or a group of covalently-bound atoms such as a methyl or an ethyl moiety. When said molecule is a polynucleotide, said “chemical building block” corresponds to a base, or a derivative thereof, said derivative being for instance an hydroxyl-protected base. When said molecule is a polypeptide, said “chemical building block” is an amino acid, or a derivative thereof, said derivative being for instance a carboxyl-protected amino acid.

By “tag” or “tag moiety” is meant herein a specifically detectable moiety harbored by a reagent, or a target, or both. In the latter case, a moiety A of a reagent reacts with a moiety B of a target. When associated to each other, said moieties A and B form another moiety C that becomes detectable. Detectable moieties include, but are, not limited to, optical tags (e.g., fluorescent moieties), electrical tags that can be detected electrochemically (for instance, ferrocenes and quinones), mass tags that are labels giving specific mass spectrometry signature peaks, and the like.

The present invention relates to a microfluidic device for nucleic acid and/or protein multiplex analysis, wherein said device comprises a capillary on the inner surface of which is attached an array of at least two reagents.

As defined above, the number of reagents attached to the surface of said capillary is comprised between 2 and Nₘₐₓ.

According to one embodiment, the number of reagents attached to the surface of said capillary is comprised between 2 and about 10,000.

According to another embodiment, the number of reagents attached to the surface of said capillary is comprised between 2 and about 100.

More specifically, each reagent occupies a definite area of said surface. According to one embodiment, said specific area is a section of said surface (for instance, a cylindrical section; see FIG. 1).

The microfluidic device according to the invention can comprise, but is not limited to, an array of oligonucleotides, peptides, polysaccharides, and other chemicals and reagents that can be attached to the inner surface of the capillary.

In one embodiment, the reagents contained in the array are oligonucleotides that act as probes in a multiplex hybridization analysis (see Example 3-B below).

According to one aspect of said device, fluid flow in the capillary is pressure-induced.

According to other aspects, fluid flow is, for instance, electro-kinetically induced, and/or induced by shear forces, gravimetrical forces, capillary action, and the like.

In the microfluidic device according to the invention, the single capillary can act as a flow reactor for immobilization (Example 1) or in situ synthesis (Example 2) of the above-mentioned array of reagents, and/or as an analyzer of nucleic acids and/or proteins. According to an embodiment, these steps, i.e., the immobilization or in situ synthesis step (hereafter referred to as the “synthesis step”) and the analysis step, are serially repeated in such a way that multiplex assays are performed in series in the. microfluidic device.

Moreover, as far as most biochemical analyses require the temperature to be precisely controlled, the microfluidic device according to the invention can further comprise a system to control temperature in the capillary. As an example, when PCR is used to amplify target DNA, the device temperature is usually cycled between 40 and 95° C. Systems were developed and are commercially available to enable PCR to be performed in capillaries. Such a system, for instance the Lightcycler from Roche Diagnostics (F. Hoffmann-La Roche Ltd, Basel, Switzerland) (PCR in capillaries), the P/ACE™ Series system from Beckman Coulter, Inc. (Brea, Fullerton, Calif., USA) (capillary electrophoresis with thermostated capillary compartment), can be adapted by the skilled person to perform temperature-controlled experiments (not only PCR) in a microfluidic device as described herein.

The present invention also concerns a capillary, alone or comprised in the microfluidic device described above, said capillary containing an array of at least two reagents that can be used to perform a plurality of biochemical analyses (see FIG. 1).

In one embodiment, the diameter of a capillary according to the present invention is between about 200 and about 500 μm. In another embodiment, said diameter is between about 10 and about 500 μm. In yet other embodiments, said diameter is between about 10 and about 200 μm, or between about 1 and about 200 μm.

Some dimensions, surface areas, and volumes of capillaries according to the present invention are given in the following Table 1. The values listed herein are merely illustrative and do not limit the scope of the invention.

TABLE 1

| Diameter (μm) | Length (μm) | Surface (μm²) | Volume (nl) |
|---|---|---|---|
| 10 | 10 | 314 | 0.0008 |
| 10 | 50 | 1571 | 0.0039 |
| 10 | 100 | 3142 | 0.0079 |
| 10 | 1000 | 31416 | 0.0785 |
| 50 | 10 | 1571 | 0.0196 |
| 50 | 50 | 7854 | 0.0982 |
| 50 | 100 | 15708 | 0.1963 |
| 50 | 1000 | 157080 | 1.9635 |
| 100 | 10 | 3142 | 0.0785 |
| 100 | 50 | 15708 | 0.3927 |
| 100 | 100 | 31416 | 0.7854 |
| 100 | 1000 | 314159 | 7.8540 |
| 200 | 10 | 6283 | 0.3142 |
| 200 | 50 | 31416 | 1.5708 |
| 200 | 100 | 62832 | 3.1416 |
| 200 | 1000 | 628319 | 31.4159 |

In one embodiment, the capillary of the invention can be either flexible or rigid. In another embodiment, said capillary is rigid. For instance, said capillary is made of glass, as illustrated in Example 1 and Example 2 (see below).

The capillary according to the present invention enables attachment, on the inner surface and along the length thereof, of “functionalizable” sites. By this way, chemical moieties can be immobilized (Example 1) or built up (Example 2), forming, once completed, an array of reagents on said surface.

Said “functionalizable” sites are constructed on said inner surface using photochemical methods. Photochemical reactions can be initiated by irradiation. Irradiation sources include, but are not limited to laser light, CRTs, LEDs, Resonant Microcavity Anodes, photodiodes, broad wavelength lamps, and the like. Irradiation can be focused to discrete sites along the capillary length using optical or physical methods. Irradiation can also be accomplished in multiple sites, in parallel or in series. The photochemical reactant sites can then be serially modified by flowing various reacting species in the flow-through capillary. In this way, said capillary is a flow reactor that behaves as a synthesizer and enables numerous and various chemical moieties to be attached along its length.

Two methods for preparing a capillary according to the invention can be used.

The first method comprises the preparation of a linear array of reagents on the surface of a solid substrate. The substrate is then bonded to a patterned second substrate to create a capillary around the linear array.

The second method relates to the preparation of a linear array of reagents inside a capillary, either by immobilizing pre-synthesized reagents (Example 1) or by synthesizing in situ said reagents (Example 2), respectively.

Said second method forms part of the present invention. Thus the invention relates to a method for attaching reagents on the inner surface of a capillary.

A first embodiment of said method comprises:

a) “functionalizing” said surface using a linker modified with a removable protective group;

b) generating free reactive moieties on specific areas of said surface by selectively “deprotecting” said linker;

c) sequentially immobilizing pre-synthesized reagents on said moieties; and

d) repeating steps b) and c) until all said reagents are attached.

In a second embodiment, the method for attaching reagents on the inner surface of a capillary comprises:

a) “functionalizing” said surface using a linker modified with a removable protective group;

b) generating free reactive moieties on specific areas of said surface by selectively “deprotecting” said linker;

e) introducing a chemical building block in said capillary;

f) discarding said building block which is in excess; and

g) repeating steps b), e), and f) until all said reagents are built up.

In the method of attachment described herein, a photochemical process is provided to spatially direct the preparation steps at specific areas of the inner surface of the capillary.

Said capillary is thus made of optically clear material, such as glass or quartz, allowing the light to cross the capillary walls and initiate reactions therein.

In step a), the inner surface of the capillary is “functionalized” using a synthetic linker. Said linker is modified with photo-chemically removable protective groups (see Example 2-A).

In step b), “deprotection” of the linker is mediated by light irradiation. Light is directed through the capillary at specific areas, in order to selectively “deprotect” the linker and to create localized attachment sites for the reagents.

To do so, a laser light can be used.

Importantly, the “deprotecting” irradiation in step b) has to be spatially selective, i.e., directed at one or more specific areas of the capillary surface.

The number of different compounds attached on a single array is limited by the length of the capillary, and the achievable resolution of the lithographic step. Moreover, during each irradiation, several sections of the capillary can require to be exposed.

Different methods are described in the literature for producing collimated light beams that can be useful for the method of attachment of the present invention. According to these methods known by the skilled artisan, means that can be use include, for instance, individual lasers, masks, arrays of mirrors, and TV screens.

According to one aspect of the method of attachment of the invention, the wavelength of the directed light used in step b) is greater than about 350 nm. Said wavelength is for instance 365 nm.

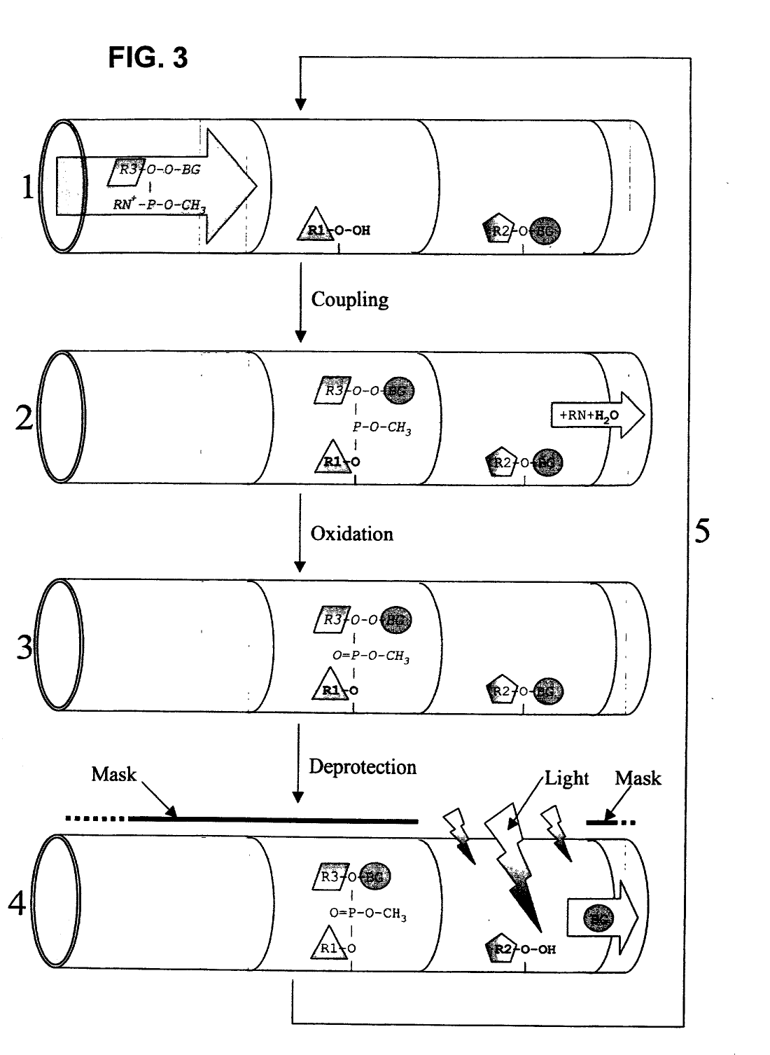

According to the first embodiment of the method of attachment described above [steps a) to d)], pre-synthesized compounds that can be used include, without limitation, drug candidates, oligonucleotides, cDNAs, proteins, polysaccharides, and the like (see FIG. 3).

During step b), light is directed orthogonally at the first section of the capillary in order to release the protective group from the linker and expose said linker in its reactive form.

In step c), the first compound is injected in the capillary and the reaction of attachment occurs selectively in the section that was irradiated in the preceding step [i.e., in step b)]. Immobilization can be performed on said section through covalent binding (see FIG. 3, step 1 wherein R1-O—OH can react with the reagent moiety that is injected).

Light is then directed to the next section of the capillary (FIG. 3, step 4 wherein BG is released from R2-O-BG), and the next compound is applied. This cycle is repeated according to step d) until the array is created.

According to the second embodiment of the method of attachment described supra [steps a), b), and e) to g)], reagent in situ synthesis permits to combine different strategies to finally create, in a few steps, arrays of large number of reagents (Examples 2-B and 2-C below).

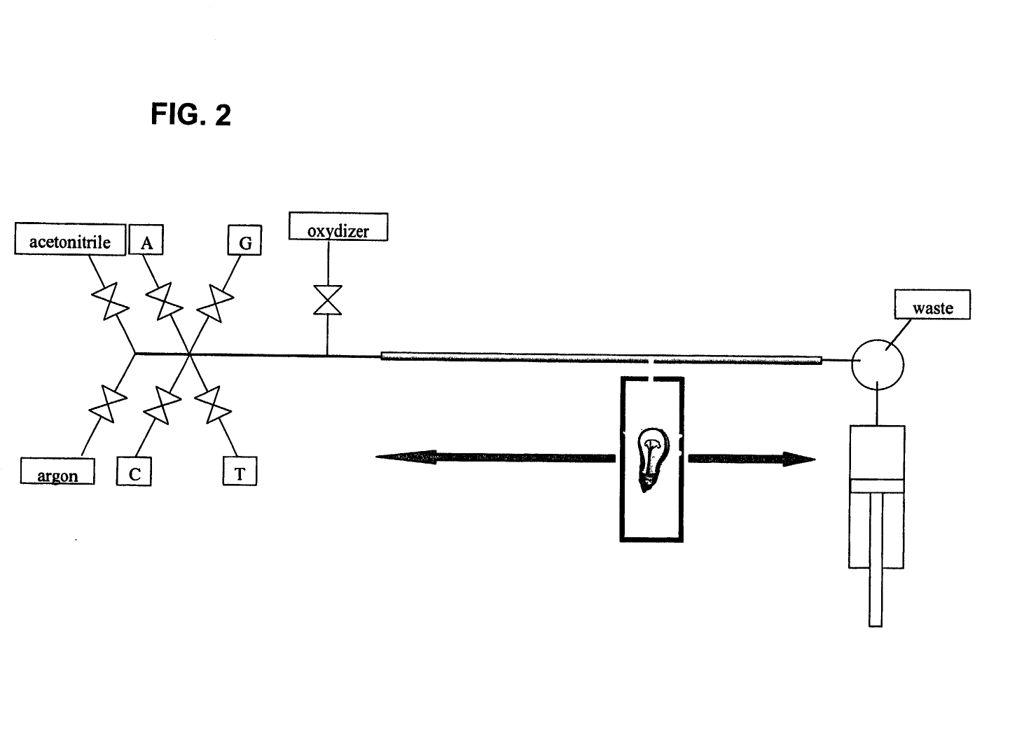

According to one aspect of said embodiment, reagents used therein are oligonucleotides, and chemical building blocks used in steps e) and f) are for example hydroxyl-protected deoxyribonucleoside phosphoramidites (see FIG. 2 and Example 2-B).

According to said aspect, in step a), the surface of the capillary made of optically clear material such as glass, is “functionalized” with a chemical group, for instance a 3-ethoxy-aminopropyl-silanol group, from which will be extended the oligonucleotide chain terminating with a photo-chemically removable protecting group (Example 2-A below) (7).

Orthogonal light is then directed at specific cross-sections of the capillary to produce localized photo-deprotection [step b)]. Several sections can be simultaneously or successively irradiated to provide reactive sites for nucleotide attachment.

According to step e), the first one of a series of chemical building blocks, for instance hydroxyl-protected deoxyribonucleoside phosphoramidites (A, G, T, C), is introduced in the capillary, and chemical coupling occurs selectively in the sections of the surface that were previously irradiated (FIG. 2).

During step f), reagent which is in excess is pushed outside the capillary.

Light is then directed at different sections of the surface of the capillary and the chemical cycle is repeated until all desired oligonucleotides are assigned to a defined section of the capillary [step g)] (8, 9).

Using such a method of in situ synthesis, a number X of oligonucleotides of length N can be synthesized in 4×N cycles.

The present invention departs from both microarray and microfluidic technologies, in that it enables an enzymatic, a genetic, and a proteomic high throughput and/or multiplex analysis to be performed using a flow-through system that can incorporate a myriad of chemical reagents.

Reagents are immobilized or synthesized in situ when the capillary acts as a flow-through reactor. Then, the capillary acts as a flow-through analyzer, allowing serial detection of single or multiple species having perturbations in the parameter of detection of the flow caused by tags.

The capillary allows multiplex detection of different tags released simultaneously, or detection of one tag released in time or of different tags released at different times.

Tags can be sequentially released for detection (analysis step) or during the synthesis step.

The present invention thus relates to a method for analyzing nucleic acids and/or proteins, said method enabling a multiplex analysis to be performed, and comprising:

h) introducing a sample into a microfluidic device according to the invention;

i) allowing said sample to react with all or part of the reagents attached to the inner surface of the capillary contained in said microfluidic device; and

j) detecting light emission using an excitation light source associated to a detector.

Samples that can be used according to this method include, but are not limited to genomic DNAs, mRNAs, proteins, cell and tissue extracts.

One of the more frequent limitations of high throughput biochemical analyses is the quantity of sample that is available for each analysis. There is therefore a crucial need to minimize sample intake. A capillary as described above is thus very suitable in that regard because a very small volume of sample injected therein is sufficient to enable an analysis to be performed.

Capillary dimensions can be selected to minimize sample volume to inject therein. Moreover, dimensions of each localized section of said capillary to which a specific reagent is assigned can be determined using the method of attachment described above. It is therefore possible to deduct the number of different compound per linear array in a capillary according to the present invention.

Thanks to the above method of analysis, volumes of sample are less than about 1 μl. In a first embodiment, said volumes are between about 1 and about 500 nl. In other embodiments, said volumes are between about 0.1 and about 50 nl, or between about 1 and about 500 pl. Examples of volumes of sample that are required depending on the parameters of the capillary used are given in Table 1 (supra).

Since such small volumes of sample are required, the concentration of the target(s) in said sample can be higher than when utilizing other methods that do not use capillaries. Therefore, the devices and methods of the present invention permit to achieve higher sensitivity levels. Said devices are thus useful when the sample is available in very small amounts, such as when working with extracts obtained from a single cell.

On the contrary, high volumes of sample can also be treated using the devices and methods of the invention. In this case, sample volumes can be higher than about 1 μl. Said volumes can be as high as about 99 ml. According to one embodiment, said high volumes of sample are comprised between about 1 μl and at least about 10 ml. In this respect, a continuous flow of sample is introduced during step h) in the microfluidic device. Detection according to step j) is thus also performed continuously.

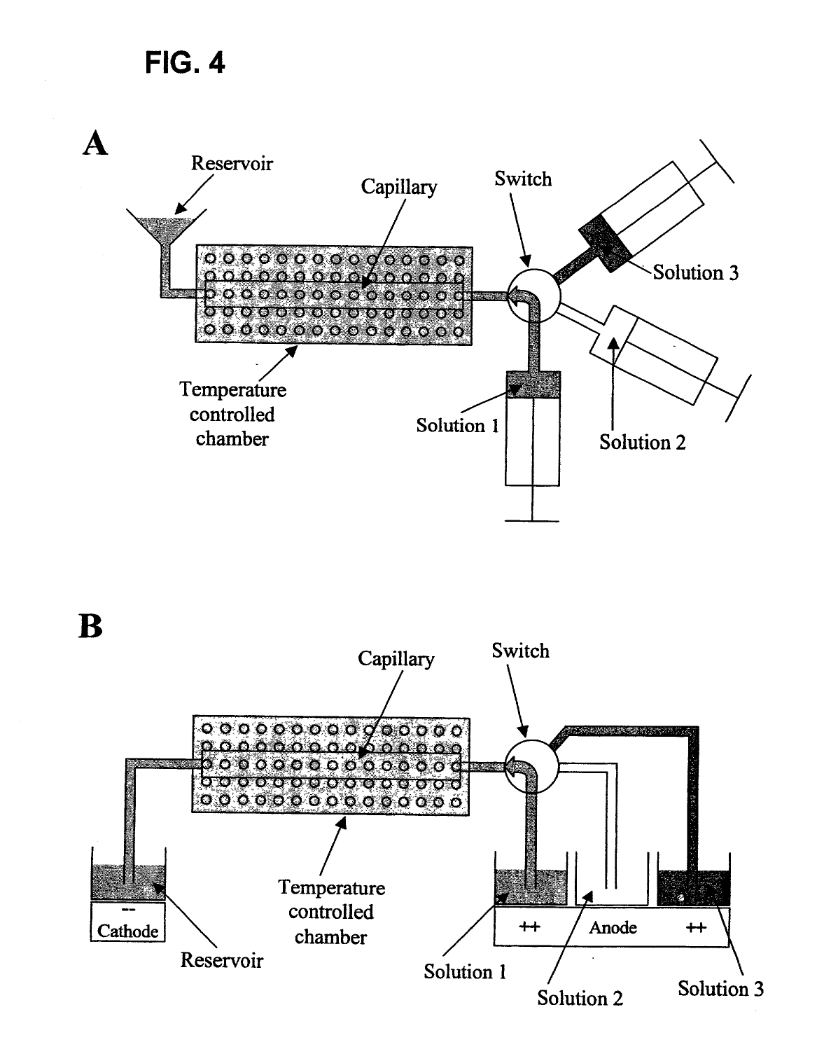

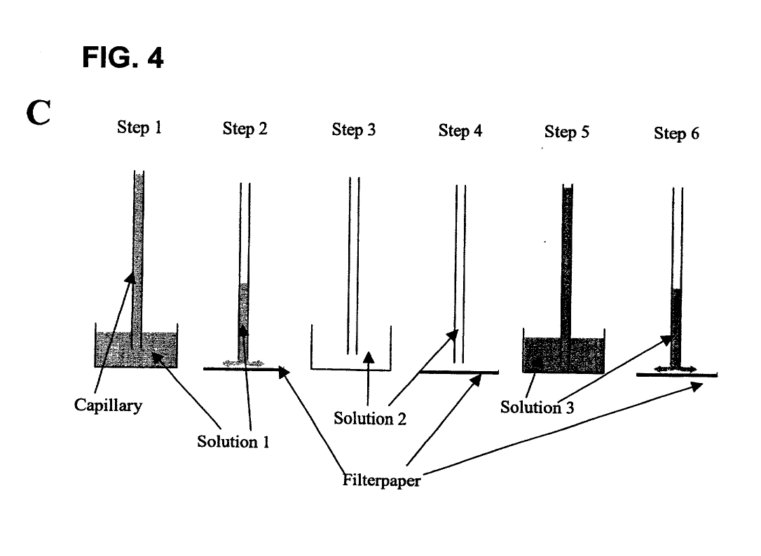

Different modes of sample introduction are described hereafter for illustrative purposes (FIG. 4). This enables the person skilled in the art to select, not only among these modes but also among other modes he knows, the most appropriate one to achieve his goal, this selection depending on the nature of the analysis to perform, the availability and chemical structure of the sample that is used, and the detection strategy that is chosen.

The sample can be introduced in the capillary using a pressure-driven syringe pump (FIG. 4 A). If the sample volume is less than the capacity of the capillary containing the linear array, then the volume and the linear flow rate will determine the time spent by the sample in contact with the surface-bound reagents. If the available volume is sufficient to cover the entire array, then the flow can be interrupted to provide an adequate incubation time.

By reversing the pressure, the direction of the flow can also be changed. Alternating flow during incubation generates a mechanical agitation, allowing a better exposure of the target(s) to the reagents bound on the surface of the capillary.

The skilled artisan is aware of the fact that pressure injection devices are commercially available, having been developed for numerous automates [computer controlled syringe pumps by Carvo (Applied Biosystems, Foster City, Calif., USA), Hamilton Company (Reno, Nev., USA), Tecan Group Ltd. (Männedorf, Switzerland) P/ACE™ Series system from Beckman Coulter Inc.]. A wide range of volumes and flow rates are thus achievable.

With very small amounts of sample (for instance less than about 100 nanoliters) and/or when a precise control of injected amounts is required, electro-kinetic injection can be used (FIG. 4 B).

Also, capillary forces can be used to introduce or discard sample into or from capillaries (FIG. 4 C). Such a process can be easily automated using robotic systems that will facilitate and reduce the handling of all capillaries used herein.

According to the method of analysis of the invention, the sample is applied to the array by injection [step h)], and the reaction with each reagent obtained during step i) is detected across each corresponding section of the capillary [step j)].

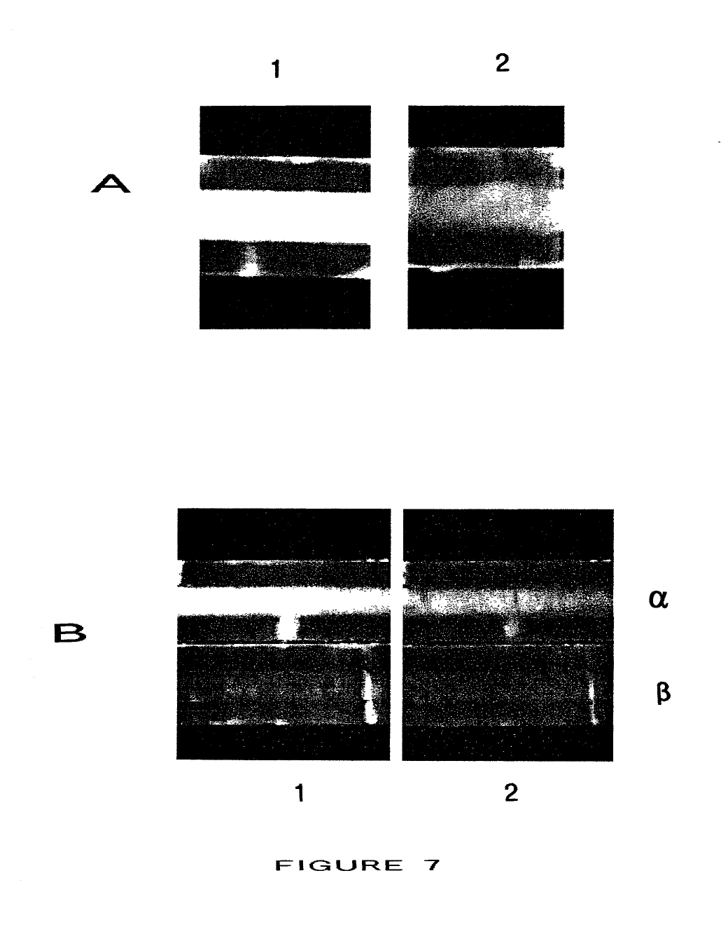

In said step i), the sample is let to react with all or only some reagents contained in the microfluidic device. As set forth in the Examples below, said step i) can comprise hybridization of the sample to those reagents that are complementary thereto, said reagents acting as probes (see Example 1, FIG. 7 B, and Example 3-B hereunder).

Several methods have been developed to detect biochemical reagents in capillaries. Detection in step j) can be performed by methods including, without limitation, photochemical, electrochemical, electrophoretic, fluorescent, UV/NIS absorbance, MS, IR, and/or chromatographic methods.

Optical detection methods are usually used since they alleviate the problem posed by the necessity to detect the target at the capillary end. Suitable methods are, for instance, UV-VIS spectrophotometry, direct fluorescence, and time-resolved fluorescence. Direct fluorescence combines high sensitivity and practicality, and it is thus useful to detect target compounds inside a capillary.

In one embodiment of the above method of analysis, detection in step j) is thus performed by direct fluorescence.

At the time, no instrument exists to scan a capillary lengthwise. However, the invention circumvents this lack in the art by enabling a signal to be detected thanks to relative mobility of the capillary and the detector.

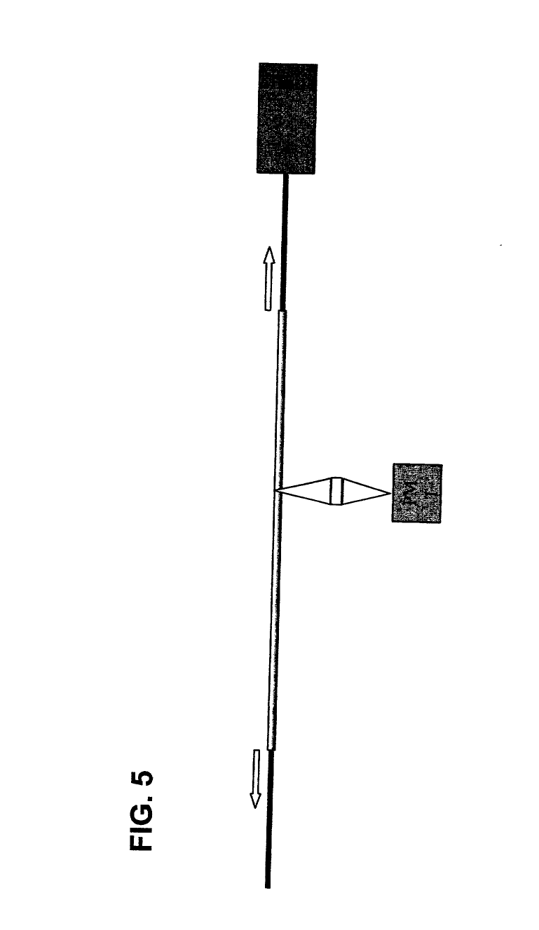

In one embodiment of the method of analysis of the invention, the microfluidic device is moved whereas the excitation source is fixed, and detection in step j) is thus performed at a single point of said device (FIG. 5).

In another embodiment, said excitation source is moved whereas said device is fixed, and detection is performed along the length of said device.

In yet another embodiment, the device is entirely light-excited, and the whole emitted light is detected at once.

According to such embodiments, a tag is released in the capillary when the sample reacts with the reagent, so that said tag can be directly detected.

In another embodiment, the device of the invention is associated to an additional analyzer device, such as a capillary electrophoresis device, a mass-spectrometer, a spectrophotometer, and the like. By this way, an organized stream of separated tags can be detected by successive injections of the fluid obtained at the end of the capillary in such an additional analyser device.

In one embodiment of the method of analysis according to the invention, a fluorescently-tagged nucleic acid sample is injected into the capillary [step h); see Examples 1-D and 3-B]. Said sample hybridizes to reagents located therein thanks to the presence of complementary oligonucleotides [step i)]. After washes to remove all the unbound fluorescent material, a laser excitation enters through one end of the capillary and excites all the bound fluorescent molecules along the capillary [step j)]. Fluorescent emission is collected through both the wall of the capillary and an optical filter by a sensitive detector such as a PMT or a scanner or a linear charged coupled device (CCD). A quantitative fluorescent image of the capillary array can be generated and used for analysis purpose.

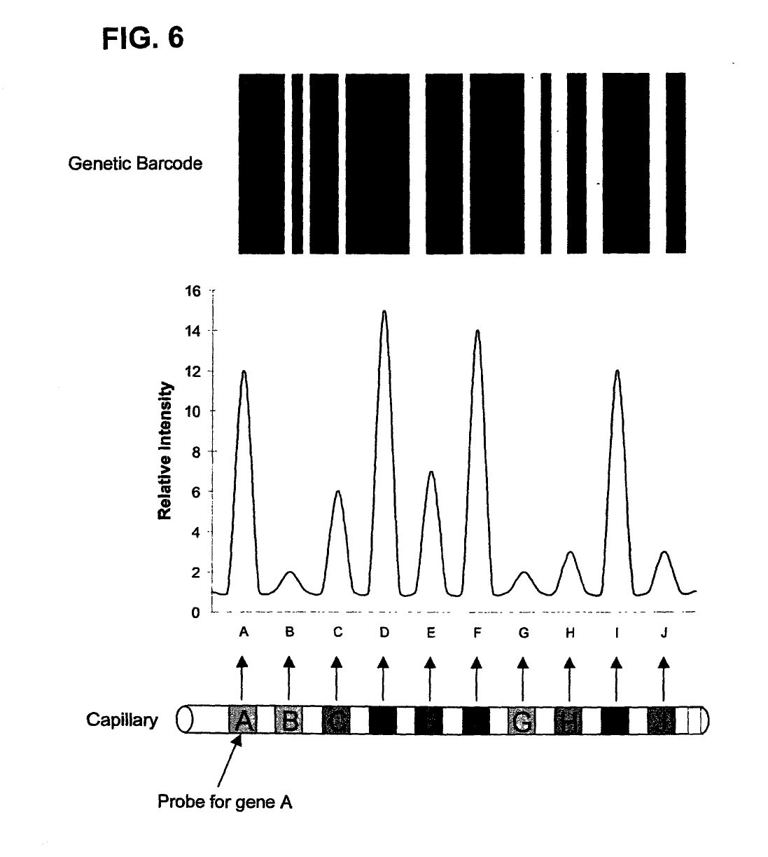

As shown in FIG. 6, the output graphically represents the relative intensity from one probe to another. Areas under the curve thus obtained can be integrated and, by the use of appropriate controls and reference probes (see Examples 3-C, 3-D, and 3-E), ratios between outputs obtained from two different reagents can be determined.

According to one aspect of the method of analysis described herein, the output is a barcode specific to the sample, as illustrated in FIG. 6.

Said barcode also forms part of the present invention.

Reagents attached to the inner surface of the capillary can remain permanently attached after the synthesis phase.

Alternatively, said reagents can be released if and when desired.

The method of analysis described herein can thus further comprise a step of releasing reagents from the inner surface of the capillary contained in the microfluidic device, once said reagents have reacted with the sample.

According to this embodiment, the present invention concerns a method for analyzing nucleic acids and/or proteins, said method enabling a multiplex analysis to be performed, wherein said method comprises:

h) introducing a sample into a microfluidic device according to the invention;

i) allowing said sample to react with all or part of the reagents attached to the inner surface of the capillary contained in said microfluidic device;

k) releasing the sample-reagent interacting products formed in step i) from said inner surface of said capillary; and

j) detecting light emission using an excitation light source associated to a detector.

In this case, detection step j) is performed outside the capillary contained in the microfluidic device, using means as described above.

Released sample-reagent interacting products can thus act as reporters in an enzyme activity assay, as well as in genotyping, reaction kinetics, and the like.

Released sample-reagent interacting products can also be used according to a feedback mode, to enable changes in the flow rate to be detected during analysis of tags, so that said changes in the flow rate can be accounted for during the analysis process.

As indicated above, detection can be performed once, outside the capillary [step j)]. In another embodiment, detection can be performed twice, either simultaneously or successively, within the flow stream and outside the capillary. For example, a first detection step is performed within the flow stream, either during the reaction step i) or once said reaction step i) is completed; and a second detection step is performed as the effluent leaves the capillary, on released sample-reagent interacting products.

As set forth in the following Examples, applications of devices and methods according to the present invention include, but are not limited to, polymorphism and mutation analysis, mRNA characterization and/or quantification, as well as protein function analysis, drug screening, and the like.

### EXAMPLES

The following Examples are merely intended to be illustrative and should not be interpreted as limiting the scope of the present invention.

### Example 1

### Preparation of a Capillary on the Surface of Which is Attached an Array of Presynthesized Probes

### 1-A: “Functionalization” of a Capillary

Capillaries are purchased from SGE (Milton Kaynes, UK). The inside diameter is 100 μm and the diameter including the inner wall but excluding the polyimide jacket is 300 μm. These capillaries are in silica transparent to UV.

Capillaries were cleaned using a NaOH solution (3.6N) in ethanol 50%, and then washed with bi-distilled water. They were dried using a stream of air, and treated with a solution of γ-aminopropylsilane 5% in absolute ethanol. Reaction was performed overnight. Capillaries were rinsed, first with ethanol 95%, then with bi-distilled water, then again with ethanol 95%. The silane layer was reticulated by baking the capillaries at 110° C. during 3 hours. Capillaries were immediately filled using a solution of glutaraldehyde 10% in water. Reaction was performed during 2 hours and capillaries were rinsed with bi-distilled water.

### 1-B: Attachment of Oligonucleotide Probes

Two oligonucleotides (17 and 19 mers) harboring a primary amine function in 5′ position were synthesized [CP—NH2: NH2-CCTTGACGATACAGCTA (SEQ ID No. 1); and 261A-NH2: NH2-GCCACATCGCTCAGACACC (SEQ ID No. 2)]. They were diluted at a concentration of 50 μM in phosphate buffer 150 mM, pH 8.5. Each probe was introduced in a different capillary. Reaction was performed during 1 hour and a half at room temperature.

### 1-C: Capillary Post-treatment

In order to saturate the sites that did not react, capillaries were treated during 20 minutes at 50° C. using a blocking solution (50 mM ethanolamine, 0.1 M Tris, pH 9.0, SDS 0.1%) heated at 50° C. Capillaries were first rinsed using SSC 4×-SDS 0.1%-containing buffer heated at 50° C., then using bi-distilled water. After drying, capillaries were ready to use.

### 1-D: Hybridization of the Capillary-attached Probes to a Fluorescent Oligonucleotide Target

An oligonucleotide target, 5′-labelled using a fluorescent molecule named Cy3, complementary to the CP-NH2 probe, was synthesized: CPc-Cy3 [Cy3-TAGCTGTATCGACMGG (SEQ ID No. 3)]. It was diluted in hybridization buffer (PBS 1×, NaCl 0.5M, EDTA 10 mM, salmon sperm DNA 100 μg/ml, formamide 50%) to obtain a concentration of 25 μM.

This solution was used to fill both capillaries, in which was attached either CP-NH2 or 261A-NH2. Hybridization was carried out for 1 hour and a half at 42° C. In order to eliminate as much unhybridized fluorescent target as possible, capillaries were rinsed using a solution containing SSC 2× and SDS 0.1%.

Capillaries were then observed in this solution using a fluorescence microscope.

### I-E: Hybridization Observation

Fluorescence emitted by the capillaries was observed using an Olympus microscope supplied with filters for Cy3 (λexcitation filter=550 nm; λemission filter=570 nm). The magnifications used were 10× and 20×. The image-analyzing software was Analysis, and the data-acquiring time was 20 or 50 millisecondes. An illustration of the obtained images is given in FIG. 7.

### Example 2

### Preparation of a Capillary on the Surface of which is Attached an Array of in Situ-synthesized Reagents

### 2-A: “Functionalization” of a Capillary

A glass capillary is cleaned by successively injecting a Nanostrip solution (Cyantek, Fremont, Calif.), 10% aqueous NaOH and then 1% HCl, and rinsing thoroughly between each step with deionized water. After the last wash, the capillary is dried with a stream of nitrogen.

The internal surface of the capillary is uniformly functionalized in gas phase with dimethyl aminopropylsilane and free amino groups, forming a monolayer bound to the glass with a C3 spacer, are protected with a nitroveratryloxycarbonyl (NVOC) group which can be removed photo-chemically (7). The capillary is washed with acetonitrile and dried with a stream of nitrogen.

### 2-B: Attachment by in situ Synthesis of Polynucleotide Probes Within the Capillary

Phosphoramidites are used at a concentration of 50 mM in dry acetonitrile and coupling reactions are performed on a custom-built capillary array synthesizer, corresponding to an automated exposure device and a capillary holder connected to a PerSeptive Expedite DNA synthesizer (Applied BioSystems, Foster City, Calif., USA). Reagent delivery is controlled by the standard programmed coupling procedure provided with the Expedite instrument, except that minor adjustments are made to: (i) eliminate the detritylation step and the exposure pause; and (ii) take into account the fact that the flow rate changes and that reagent volumes used are smaller than those for which said procedure was first conceived. The program also includes the sequence and masked positions for each nucleotide probes.

Light is projected from a 500W mercury arc lamp through a mask. An exposure time of 1 min at λ=365 nm and 120 mW/cm2 is required. Shorter exposures can also be applied using an argon laser or a cathode-ray-tube (CRT) type device, i.e., a TV screen.

5′- NVOC protected 3′-cyanoethyl phosphoramidites are used as monomeric building blocks (see FIG. 2). Rapidly removable base protecting groups such as phenoxyacetyl (PAC) for A and G, and isobutiryl (iBu) for C, are used so that the final “deprotection” of the capillary array can be carried out under mild conditions to ensure that, doing so, oligonucleotides are not cleaved from the surface of the capillary.

The first amine-coated sections of the capillary are selectively activated by directing the light through the mask. The irradiated amine groups react with the 5′-NVOC protected T phosphoramidite previously activated by tetrazole. Once the reaction is completed, reagents in excess are flushed out from the capillary. Steps of irradiation and base addition are then repeated using 5′-NVOC protected A(PAC), G(PAC), and C(iBu) phosphoramidites. The synthesis cycle is finally completed as in the usual practice by oxidation and capping. Said synthesis cycle is repeated until all the polynucleotides are synthesized. The last irradiation removes the 5′-NVOC protective groups from all said polynucleotides. The capillary array is then deprotected in a 50% solution of ethanolamine in ethanol for 1 hour at room temperature, rinsed with water, and dried using a nitrogen stream.

### 2-C: Attachment by in situ Synthesis of Deptide Probes Within the Capillary

Amino acids activated in the form of their 1-hydroxybenzotriazol (HOBt) ester derivatives, protected by NVOC, are used as monomeric building blocks (7). At the end of synthesis, other deprotecting steps are necessary to free the side chains of the peptides. For instance, removal of the trytil protective groups is achieved by adding a 94:1:5 dichloromethane/trifluoroacetic acid/triisopropylsilane solution in the capillary for 2 minutes. The solution is then removed by applying nitrogen pressure. Since many different protective groups can be used for peptide synthesis purposes, many different deprotection procedures can be followed.

### Example 3

### Use for Detecting Polymorphisms and Alleles of the NAT2 Gene

### 3-A: Background

Polymorphisms in the NAT2 gene encoding the N-acetyltransferase 2 have been associated with slow, intermediate, and fast metabolism of isoniazid, hydrazaline, and procainamide. This enzyme is also regarded as being responsible for the inactivation of aromatic and heterocyclic amine carcinogens.

The coding region of the NAT2 gene spans 872 base pairs (GenBank Accession No. NM-000015). Mutations in the NAT2 gene, occurring singly or in combination, define numerous alleles associated with decreased function. Said alleles determine the efficiency (rapid or slow) of drug metabolism, and also influence susceptibility to colorectal and bladder cancer.

These specific alleles are listed in the following Table 2 (16). Said Table describes the seven most common single nucleotide polymorphisms (SNPs) found in the NAT2 gene (G191A, C282T, T341C, C481T, G590A, A803G, and G857A), and defines the nine most common alleles (*4 has been defined as the wild type allele). The amino acid changes resulting in some cases from the SNPs are also set forth in Table 2 hereunder.

TABLE 2

| | SNP | | | | | | | | |
|---|---|---|---|---|---|---|---|---|---|
| Allele | G191A | C282T | T341C | C481T | G590A | A803G | G857A | PH | FR |
| *4 | G | C | T | C | G | A | G | Rapid | 23.4 |
| *5A | G | C | C | T | G | A | G | Slow | 2.5 |
| *5B | G | C | C | G | G | G | G | Slow | 40.9 |
| *5C | G | C | C | C | G | G | G | Slow | 2.6 |
| *6A | G | T | T | C | A | A | G | Slow | 28.4 |
| *7B | G | T | T | C | G | A | A | Slow | 2.1 |
| *12A | G | C | T | C | G | G | G | Rapid | 0.1 |
| *14A | A | C | T | C | G | A | G | Slow | Rare |
| *14B | A | T | T | C | G | A | G | Slow | 0.1 |
| AA | R → Q | None | I → T | None | R → Q | K → R | G → E | | |
| Change | | | | | | | | | |

PH, phenotype; FR, frequency; AA, amino acid.

NAT 2 provides a low complexity model for developing an assay for detecting polymorphisms and alleles. Its clearly defined and well-documented genetics makes such an assay prototypical. Each of the seven polymorphisms listed in Table 2 is a marker for more than one NAT2 allele and each variant allele is defined by more than one SNP substitution.

In the assay described below, typically homozygous and heterozygous genotypes are determined for each polymorphic site and, then, probable allele assignments are made. This ensures and improves the accuracy of the assay.

### 3-B: Hybridization Assay for Detecting Polymorphisms and Alleles of the NAT2 Gene

A PCR product is amplified from human genomic DNA containing the NAT2 gene. More specifically, primers 5′-GTCACACGAGGAAATCAAATGC-3′ (SEQ ID No. 4) and 5′-GTTTTCTAGCATGMTCACTCTGC-3′ (SEQ ID No. 5) are used to amplify 1.2 kb of genomic DNA that contains the NAT2 gene (16).

This PCR product is purified using an affinity column, and nicked with DNase into smaller fragments, of an average size of approximately 100 bp. These short fragments are end-labeled with biotin-ddATP or with fluorescently-tagged dd-UTP during a terminal transferase reaction. Prior to hybridization, these labeled fragments (representing the targets) are denatured for 5 minutes at 95° C. and quickly chilled on ice for 5 minutes.

A capillary is prepared as set forth in Example 2-B using the oligonucleotides set forth in Table 2 of Cronin et al. (16) as probes.

The capillary is pre-washed in 2× SSC, 1 mM EDTA, and 0.1% Tween 20 for 5 minutes at room temperature.

Hybridization between targets and probes is performed in 1×2-morpholinoethanesulfonic acid (MES) for two hours at 45° C. After hybridization, washing solutions (2× SSC and 0.1% Tween 20 at room temperature (RT) for 10 minutes, followed by 0.1× SSC and 0.1% Tween 20 at RT for 10 minutes) are injected. If biotin is used as a label, targets are stained with a Cy3-streptavidin conjugate.

The above hybridization protocol shall be considered as being a general guideline since it can be adjusted to be suited to different labels including, but not limited to biotin, Cy3 and Cy5 tagged NTPs, amino-allyl labels, and radioactive labels such as 33P, as well as to different targets, such as DNAs and RNAs, and to different probes, different probe lengths, and different capillaries.

### 3-C: Use of Internal Controls

To analyze hybridization performance, intensities at complementary probe sites are compared to each other. In addition to perfectly matching control probes, single- and double-mismatching probes are included. Labeled targets complementary to these control elements are added either separately or within the hybridization mixture at known quantities.

An ideal result is when perfectly matching controls display high intensities and maximal discrimination compared to the mismatching controls.

### 3-D: Use of Characterized Hybridization Samples

To evaluate different capillary designs and probe sets for their performance in discriminating NAT2 genotypes, a set of genomic DNA samples with known NAT2 genotypes is tested. These samples include the *4 wild-type (wt) homozygote genotype, as well as samples that collectively represent each of the seven common polymorphisms that lead to heterozygote organisms.

To ensure correct genotyping, these samples are dideoxy-sequenced. Samples from this genomic DNA collection, as well as other characterized DNA samples, can be used during all the steps described herein to optimize capillary performances.

### 3-E: Iterative Design of Polynucleotides for Detecting Polymorphisms and Alleles

Sequence environment and sequence composition determine DNA-DNA duplex stability.

Calculated thermal melting points for the probe-target duplexes are used for initial probe design and a common melting temperature (Tm) is chosen. Tm can be calculated according to the formula Tm=81.5+(100×0.41×percent GC)−(675/length). Other algorithms can be used to calculate Tm, said algorithms being well-known by the skilled person.

The polymorphism site is generally centered in the probes and the probe length can vary as needed to match the targeted Tm value. The aim is at designing a probe capable of detecting specific genotypes by hybridization. To do so, it is generally necessary to maximize the fluorescence intensity obtained with exact complementary probes, compared to that given by negative mismatching controls (see Example 3-C). Therefore, it can be necessary to emphasize this selection criteria (i.e., the difference in fluorescence intensity depending on whether probes are matching or mismatching), using subsequent empirical design modifications of the probes to optimize the array genotyping performance.

In the context of the present invention, it is easy to design a set of probes for the sole purpose of defining which of said probes give(s) the best value for the above selection criteria, allowing the selection of the best probe(s) for subsequent final assay designs.

Using the assay described in Example 3-B and the refinements detailed in Examples 3-C, 3-D, and 3-E, the present invention appears to be well-suited to determine whether a target nucleic acid sequence hybridizes or not with the sequences used herein as probes.

### 3-F: Primer Extension Assay for Detecting Polymorphisms and Alleles of the NAT2 Gene

Instead of identifying a genotype by hybridizing a PCR product (see Example 3-B) to allele-specific probes containing perfectly internal matching (wt) and mismatching (mutant) nucleotides, allele-specific probes containing an allele-specific nucleotide at their 3′ end can be used.

These probes are attached to the capillary surface via 5′ modifications so that the 3′ end thereof is kept accessible and can be enzymatically extended. Target (such as, for example, a PCR product or genomic DNA) is added together with DNA polymerase, buffer components, and labeled deoxynucleotides (dNTPs). An enzymatic primer extension reaction is then performed to extend the probe used as a primer, utilizing the DNA target to which the probe is hybridized as a template. During said reaction, the polymerase extends the 3′ end of the probes that contain a matching (either wt or mutant if homozygote, or wt and mutant if heterozygote) nucleotide.

Alternatively, non allele-specific probes can be designed ending precisely one base upstream from the polymorphic site. In such a case, differently labeled di-deoxy nucleotide terminators (ddNTPs) replace the dNTPs in the primer extension reaction, thus permitting to add one base only, said base being located at the polymorphic site. Detection and identification of this base can be performed using a color code. Ideally, the four different ddNTPs are each labeled in a different color, the four ddNTP-associated colors being distinguished by a color-detecting device. This device can be the same device as the one used to detect the hybridization result obtained according to Example 3-B. Internal controls and characterized extension samples are used as set forth in Examples 3-C and 3-D.

### Example 4

### Patient Sample Genotyping

Results from Example 3 can be used to design an optimized assay useful for patient sample genotyping.

Such a study can be retrospective or prospective.

In a retrospective study, a number of samples from patients with a characterized phenotype are analyzed to determine a potential correlation with the genotype determined by the assay. For example, patient samples are tested for the mutations described in Example 3. These patients can have a given type of cancer. The aim of the study is thus to determine whether said type of cancer is associated to the occurrence of specific mutations in the NAT2 gene.

In a prospective study, patients that are expected to receive a given drug are genotyped before taking said drug. The occurrence of side-effects, known or unknown at that time, can be correlated with the genotype determined by the assay.

### Example 5

### Detection of mRNA Synthesis Levels

Compared to polymorphism detection, in gene expression profiling, an additional level of difficulty has to be circumvented. Indeed, an accurately report of level of expression of a gene of interest is based on numerical assessment of hybridization intensities of the target to the selected probes. Identifying the optimal probe(s) is crucial since secondary structures, as well as cross-hybridization of the probe(s) with unspecific targets need to be excluded.

As an example of probe iteration and, consequently, optimization, the beta actin gene (GenBank accession No. AB004047) can be chosen. This form of actin is a constituent of the cytoskeleton of non-muscular cells and, because of its high abundance, it is frequently used in research laboratories to normalize mRNA or gene expression profiles.

The present invention allows, but is not limited to the monitoring of the level of expression of the actin gene.

The use of oligonucleotide probes for gene expression profiling has the following advantage over the longer cDNA probes: cross-hybridizations to targets with high sequence homologies can be prevented using oligonucleotide probes. However, using such shorter probes, hybridization stringency and, therefore, efficient target discrimination is reduced.

The present invention is used to determine the optimal length of nucleotide probes. It appears that the probe length will differ among different genes or parts thereof, and it is necessary to optimize such probes accordingly. The location of the probe within the gene and its length determine the hybridization signal intensity.

To control hybridization specificity, an approach analogous to that used in Examples 3-C, 3-D, and 3-E, is used: mismatches are introduced in the center of control probes, and, for perfectly matching probes, optimal base-pairing is disrupted as it occurs. Indeed, it was observed that it was advantageous to discriminate two mismatches (mutant) from one mismatch (wt) rather than one mismatch (mutant) from a full match (wt). To do so, probes are thus designed with one arbitrary mismatch for both types (mutant and wt). Such mismatches can include either single base-pair changes, or changes of more than one nucleotide. If the signal intensity for the mismatching probes is reduced compared to the perfect matching probes, this indicates that the latter are hybridizing specifically and that hybridization conditions are chosen appropriately.

Typically, for gene expression experiments, hybridization is carried out in 1× MES buffer at 65° C. in the presence of 0.1 mg/ml herring sperm DNA and 0.5 mg/ml acetylated BSA for blocking unspecific binding sites. However, different hybridization buffer systems exist, that are well-known by the skilled artisan. The device according to the invention can use all of said systems.

The present invention can be used for iterative oligonucleotide probe design in all applications that use oligonucleotides (as set forth in Example 3-E). This includes, but it is not limited to the device according to the present invention itself, but also DNA microarrays, assays in solution, and microfluidic systems. The flexibility of said device facilitates applications such as genotyping, gene expression profiling, and others.

### Example 6

### Detection of Infectious Agents

Most of the currently available tests to detect infectious agents are indirect. For infections caused by agents such as Hepatitis C virus (HCV), Hepatitis B virus (HBV), Human Immunodeficiency virus (HIV), and other agents from both viral or bacterial origin, commonly used tests determine the presence of specific antibodies produced by the patient's immune system in response to said infectious agent. These tests are useful for blood screening, and to a limited extent, as diagnostic tools. Nevertheless, since they offer only an indirect measure of infection, they do not tell the clinicians whether the infection is past or current, or if there is a response to the therapy. An antibody-based test can also miss a recent infection, because it takes generally several days or weeks for the immune system to produce an antibody response to the infectious agent. This can be dangerous, for instance if the infectious agent rapidly spreads in the patient organism, or if the blood from an apparently healthy but recently infected person (HIV and HCV) is used for blood transfusion.

The present invention permits to elaborate tests which are based on measuring directly the presence of the infectious agent within the patient sample. These tests can detect the presence of nucleic acids of the infectious agents in the blood or in other biological samples taken from a patient, such as urine, sputum, and the like.

Two major assays are developed. The first assay is based on a PCR amplification of the infectious agent genetic material. Said assay allows the detection of at least 100 copies of said material in a single sample. PCR uses an enzymatic reaction to amplify specific nucleic acid sequences from the infectious agent when they are present in the sample. A shortcoming of this assay is that specific nucleic acid primers need to be designed from an already known sequence of the infectious agent. Therefore, if the genetic material of the infectious agent has not yet been sequenced or if it is mutating rapidly, PCR cannot be used, resulting otherwise in a false negative test.

A solution to a part of this problem posed by the PCR step, i.e., when false negatives are observed due to a lack of sensitivity (unspecific amplification), is to add a second step after said PCR step in order to analyze the result. In the context of the present invention, said second step is an hybridization step of the PCR product, obtained from the first step, to the oligonucleotide probes immobilized in a capillary (see Example 3-B). By this way, said PCR product is detected by hybridization to probe(s) specific to the infectious agent. One can thus discriminate between specific and unspecific amplification products with the hybridization step.

The second assay is based on direct hybridization of the infectious agent nucleic acid to oligonucleotide probes in the capillary of the invention. The hybridized nucleic acid is then detected through amplifications using non-enzymatic methods. The major drawback of such an assay is that it is less sensitive (minimum 1,000 copies have to be present in the sample) and it requires a larger amount of patient sample (a minimum of 1 ml blood is necessary, compared with 0.1 ml when using PCR). Nevertheless, since this test is based on hybridization only, it is not affected by minor mutations in nucleic acid sequences. Therefore, thanks to this assay, there is no risk of false-negative reaction, compared to when PCR is used.

The present invention also permits to quantify the amount of infectious agent material present in the sample, and to correlate said amount to the patient symptoms.

Such an assay can discriminate between diseases in which symptoms are very similar and when only multiple testing can reveal the causative agent. Transplant patients, AIDS patients, and the like, can be checked for opportunistic infections and treated immediately, before requiring more extensive and expensive treatments. Red Cross and other blood banks can use this test for rapid screening of blood from donors to discard contaminated blood. It can reduce the slight, but still existing risk of transferring infectious agents by blood transfusion, using blood from donors with recent viral infections.

### Example 7

### Identification of Individuals for Forensics

Variations in the mitochondrial DNA (mtDNA) control region and polymorphisms that were found on the human Y chromosome are taken into account to conceive an assay to identify individuals. Differences in the genetic make-up of individuals are detected by oligonucleotide probes directed at specific sequences, said probes being immobilized (see Example 1-B) or in situ synthesized in a capillary according to the present invention (see Example 2-B).

mtDNA is a satisfactory forensic typing locus and it is used to identify individuals based on the high diversity of profiles found in this genetic material. Human mtDNA Control Region sequences including Hyper Variable regions HV1 and HV2 were described in the literature as being useful for identification purposes. In addition, DNA polymorphisms on the Y chromosome are valuable tools for identity testing (as well as for human evolution studies). For instance, Y markers can be used in rape cases to extract male perpetrator information when a mixture of male and female DNA is present.

While the invention has been described herein in terms of the various preferred embodiments, the skilled artisan will appreciate that modifications, substitutions, omissions, and changes may be made without departing from the scope thereof. Accordingly, it is intended that the present invention is limited by the scope of the following claims, including equivalents thereof.

### REFERENCES

All of the below listed documents are incorporated herein by reference:

1. DeRisi, J. L., V. R. Iyer, and P. O. Brown: Exploring the metabolic and genetic control of gene expression on a genomic scale. Science, 1997. 278(5338):680-6.

2. Schena, M., et al.: Microarrays: biotechnology's discovery platform for functional genomics. Trends Biotechnol, 1998.16(7):301-6.

3. Pease, A. C., et al.: Light-generated oligonucleotide arrays for rapid DNA sequence analysis. Proc Natl Acad Sci U S A, 1994. 91(11):5022-6.

4. DeRisi, J., et al.: Use of a cDNA microarray to analyse gene expression patterns in human cancer. Nat Genet, 1996. 14(4):457-60.

5. Southern, E., K. Mir, and M. Shchepinov: Molecular interactions on microarrays. Nat Genet, 1999. 21 (1 Suppl):5-9.

6. Southern, E. M., U. Maskos, and J. K. Elder: Analyzing and comparing nucleic acid sequences by hybridization to arrays of oligonucleotides: evaluation using experimental models. Genomics, 1992.13(4):1008-17.

7. Kiederowski, G.: Light-directed parallel synthesis of up to 250,000 different oligopeptides and oligonucleotides. Angew. Chem. Int. Ed. Engl., 1991. 30(7):822-3.

8. Caruthers, M. H., et al.: Chemical synthesis of deoxyoligonucleotides by the phosphoramidite method. Methods Enzymol, 1987. 154:287-313.

9. U.S. Pat. No. 6,022,963, “Synthesis of Oligonucleotide Arrays Using Photocleavable Protecting Groups.”

10. U.S. Pat. No. 6,040,138, “Expression Monitoring by Hybridization to High Density Oligonucleotide Arrays.”

11. U.S. Pat. No. 6,001,311, “Apparatus for Diverse Chemical Synthesis Using Two-Dimensional Array.”

12. U.S. Pat. No. 5,837,832, “Arrays of Nucleic Acid Probes on Biological Chips.”

13. U.S. Pat. No. 6,045,996, “Hybridization Assays on Oligonucleotide Arrays.”

14. U.S. Pat. No. 6,027,880, “Arrays of Nucleic Acid Probes and Methods of Using the Same for Detecting Cystic Fibrosis.”

15. Kohara, Y.; Chen, G.; Yamaoto, T.; Okano, K.; Kambara, H., DNA hybridization analysis by beads array in a narrow capillary.

16. Cronin, M. T., M. Pho, D. Dutta, F. Frueh, L. Schwarcz, and T. Brennan. Utilization of new technologies in drug trials and discovery. Drug Metabolism and Disposition, 2001. 29(4):586-590.

1. A microfluidic device for nucleic acid and/or protein analysis, wherein said device comprises a capillary on the inner surface of which is attached an array of at least two reagents.

2. The microfluidic device according to claim 1, wherein said capillary is rigid.

3. The microfluidic device according to claim 1, wherein said capillary is made of glass.

4. The microfluidic device according to claim 1, wherein said capillary has a diameter of between about 10 and about 500 μm, or between about 1 and about 200 μm.

5. The microfluidic device according to claim 1, wherein said at least two reagents are attached to a specific area of said inner surface of said capillary.

6. The microfluidic device according to claim 1, wherein said at least two reagents are permanently attached to said inner surface of said capillary.

7. The microfluidic device according to claim 1, wherein said at least two reagents are released from said inner surface of said capillary.

8. The microfluidic device according to claim 1, wherein said microfluidic device is a flow reactor for immobilizing or synthesizing in situ said array of at least two reagents.

9. The microfluidic device according to claim 1, wherein said microfluidic device is an analyzer of nucleic acids and/or proteins.

10. The microfluidic device according to claim 1, wherein said microfluidic device serially is a flow reactor for immobilizing or synthesizing in situ said array of at least two reagents and an analyzer of nucleic acids and/or proteins.

11. The microfluidic device according to claim 9, wherein said at least two reagents are probes in a multiplex hybridization analysis.

12. The microfluidic device according to claim 10, wherein said at least two reagents are probes in a multiplex hybridization analysis.

13. The microfluidic device according to claim 1, wherein there is a fluid flow in said capillary, which is pressure-induced, and/or electro-kinetically induced, and/or induced by capillary forces.

14. The microfluidic device according to claim 1, further comprising a temperature-regulating system to control temperature in said capillary.

15. A capillary comprising an inner surface to which is attached an array of at least two reagents.

16. The capillary according to claim 15, wherein said capillary has a diameter of between about 10 and about 500 μm, or between about 1 and about 200 μm.

17. The capillary according to claim 15, wherein said capillary is rigid.

18. The capillary according to claim 15, wherein said capillary is made of glass.

19. The capillary according to claim 15, wherein said at least two reagents are attached to a specific area of said inner surface.

20. The capillary according to claim 15, wherein said at least two reagents are permanently attached to said inner surface.

21. The capillary according to claim 15, wherein said at least two reagents are released from said inner surface.

22. The capillary according to claim 15, wherein said at least two reagents are probes in a multiplex hybridization analysis.

23. A method for attaching reagents on the inner surface of a capillary, said method comprising:
a) functionalizing said inner surface of said capillary using a linker modified with a removable protective group;
b) generating free reactive moieties on said inner surface of said capillary by deprotecting said linker;
c) sequentially immobilizing pre-synthesized reagents on said free reactive moieties; and
d) repeating steps b) and c) until said reagents are attached.

24. The method according to claim 23, wherein said sequential immobilization in step c) is performed on a specific area of said inner surface of said capillary through covalent binding.

25. The method according to claim 23, wherein said removable protective group in step a) is a photo-chemically removable protective group.

26. The method according to claim 23, wherein said deprotection of said linker in step b) is mediated by light irradiation.

27. The method according to claim 26, wherein said light irradiation is a laser light irradiation.

28. The method according to claim 26, wherein said light irradiation is directed at a specific area of said inner surface of said capillary.

29. A method for attaching reagents on the inner surface of a capillary, said method comprising:
a) functionalizing said inner surface of said capillary using a linker modified with a removable protective group;
b) generating free reactive moieties on said inner surface of said capillary by deprotecting said linker;
c) introducing a chemical building block in said capillary;
d) discarding said chemical building block which in excess; and
e) repeating steps b), c) and d) until said reagents are built up.

30. The method according to claim 29, wherein said reagents are oligonucleotides.

31. The method according to claim 29, wherein said chemical building block in steps c) and d) is a hydroxyl-protected deoxyribonucleoside phosphoramidite.

32. The method according to claim 29, wherein said removable protective group in step a) is a photo-chemically removable protective group.

33. The method according to claim 29, wherein said deprotection of said linker in step b) is mediated by light irradiation.

34. The method according to claim 33, wherein said light irradiation is a laser light irradiation.

35. The method according to claim 33, wherein said light irradiation is directed at a specific area of said inner surface of said capillary.

36. A method for analyzing nucleic acids and/or proteins, said method enabling a multiplex analysis to be performed, wherein said method comprises:
a) introducing a sample into a microfluidic device according to claim 1;
b) allowing said sample to react with reagents attached to the inner surface of the capillary contained in said microfluidic device; and
c) detecting light emission using an excitation light source associated to a detector.

37. The method according to claim 36, wherein in step a), said sample has a volume of between about 1 and about 500 nl, or between about 0.1 and about 50 nl, or between about 1 and about 500 pl.

38. The method according to claim 36, wherein in step a), said sample is introduced by a continuous flow.

39. The method according to claim 36, wherein in step a), said sample is introduced using pressure, and/or electro-kinetic forces, and/or capillary forces.

40. The method according to claim 36, wherein in step b), said sample hybridizes to reagents being probes.

41. The method according to claim 36, wherein in step c), said microfluidic device is moved and said excitation light source is fixed, and wherein said detection in step c) is performed at a single point of said microfluidic device.

42. The method according to claim 36, wherein in step c), said excitation light source is moved and said microfluidic device is fixed, and wherein said detection in step c) is performed along the length of said microfluidic device.

43. The method according to claim 36, wherein in step c), said microfluidic device is entirely light-excited, and wherein the whole emitted light is detected in said detection step c) at once.

44. The method according to claim 36, wherein said detection in step c) is performed by direct fluorescence.

45. The method according to claim 36, wherein after said detection in step c), a barcode specific to said sample is obtained.

46. The method according to claim 36, wherein said nucleic acid analysis is selected from the group comprising: nucleotide polymorphism detection, mutation detection, genotyping, gene expression analysis, mRNA characterization, mRNA quantification, infectious agent detection, and individual identification.

47. The method according to claim 36, wherein said protein analysis is selected from the group comprising: protein quantification, protein-protein interaction detection, protein-nucleic acid interaction detection, protein function analysis, enzymatic activity analysis, enzymatic binding kinetics determination, and drug screening.

48. A barcode obtainable by a method according to claim 36, wherein said barcode is specific to a sample.

49. A method for analyzing nucleic acids and/or proteins, said method enabling a multiplex analysis to be performed, wherein said method comprises:
a) introducing a sample into a microfluidic device according to claim 1;
b) allowing said sample to react with the reagents attached to the inner surface of the capillary contained in said microfluidic device;
c) releasing sample-reagent interacting products formed in said step b) from said inner surface of said capillary; and
d) detecting light emission outside said capillary using an excitation light source associated to a detector.

50. The method according to claim 49, wherein said sample-reagent interacting products released in said step c) are used as reporters, and/or in a feedback mode to detect changes in the flow rate during analysis.
